(19) United States
(12) Patent Application Publication
(10) Pub. No.: US 2009/0240170 A1
(43) Pub. Date: Sep. 24, 2009

(54) SYSTEMS AND METHODS FOR DETERMINING PRE-FALL CONDITIONS BASED ON THE ANGULAR ORIENTATION OF A PATIENT

(75) Inventors: Blair R. Rowley, Xenia, OH (US); Amy R. Sipp, Beavercreek, OH (US)

Correspondence Address: DINSMORE & SHOHL LLP, ONE DAYTON CENTRE, ONE SOUTH MAIN STREET, SUITE 1300, DAYTON, OH, 45402-2023, US

(73) Assignee: WRIGHT STATE UNIVERSITY, Dayton, OH (US)

(21) Appl. No.: 12/407,044

(22) Filed: Mar. 19, 2009

Related U.S. Application Data
(60) Provisional application No. 61/038,259, filed on Mar. 20, 2008.

(51) Int. Cl. A61B 5/103

(52) U.S. Cl. 600/595

(57) ABSTRACT

A system for collecting data related to the angular orientation of a patient for use in determining the presence of pre-fall conditions may include a wireless sensor and a base unit. The wireless sensor may be attached to the patient and include a first microprocessor operatively coupled to a gyroscope and a transmitter. The base unit may include a second microprocessor operatively coupled to a receiver and a data storage unit. The gyroscope may output a signal related to the angular orientation of the patient to the first microprocessor. The first microprocessor processes the signal received from the gyroscope and transmits data related to the angular orientation of the patient via the transmitter. The receiver in the base unit receives the data from the wireless sensor and passes the data to a second microprocessor. The second microprocessor stores formats the data and stores the data in the data storage unit for analysis.

This application claims the benefit of U.S. Provisional Application Ser. No. 61/038,259, filed Mar. 20, 2008.

### TECHNICAL FIELD

The present invention relates to systems and methods for determining pre-fall conditions based on the angular orientation of a patient and, more specifically, to systems for collecting data related to the angular orientation of a patient and methods for determining the presence of pre-fall conditions based on the collected data.

### BACKGROUND

Balance difficulties are prevalent in many areas of the population. There are many reasons people lose their balance and fall down. Studies have shown that injuries related to falls have been the largest single category of hospital inpatient incident reports since the 1940s. Accordingly, various measures have been developed in an attempt to reduce or mitigate falls in populations prone to fall-related injuries such as the elderly and populations with physical impairments affecting balance.

One approach to mitigate fall related injuries has been to develop a variety of surveys and simple timing methods for predicting the risk of a fall. Unfortunately, each of these methods only work well for the particular situation and/or patient demographic that the method was originally developed for.

Other methods to mitigate fall related injuries have focused on improving the balance of populations prone to fall related injuries. For example, efforts to improve the balance and reduce the risk of falls in such populations include: the use of canes and/or walkers, implementing safety checklists, environmental modifications, and training. The use of canes and walkers have been shown to improve balance and mobility without the implement. However, such implements may also interfere with the ability of the patient to maintain balance and may require excessive physical and/or metabolic demands. Further, the use of canes and walkers is considered one of the risk factors for falling as such devices do not address or mitigate the source or cause of balance impairment which leads to falls. Similarly, the use of safety checklists and environmental modifications may be effective for eliminating the risk of falls in a familiar environment but the effect of such remedial efforts may be somewhat limited in scope. More importantly, the use of canes and/or walkers, safety checklists, environmental modifications and the like do not directly address the cause of falls, specifically balance and the loss of balance.

Measuring balance is important for diagnosing and characterizing balance impairments or disorders, providing sway feedback in balance training systems, and analyzing human motion. Some key parameters that have been quantified to characterize or measure balance include the sway distance, sway velocity, and sway acceleration of the center of mass and the center of pressure.

Balance training has been utilized to improve the balance in patients prone to falls. Balance training involves having patients stand on a force platform facing a monitor which displays sway feedback information collected from the force platform. The patient's goal is to keep a line displayed on the monitor steady or centered on the screen. This training method was used on hemiplegic patients who supported 70% of their body weight with their non-affected (healthy) leg. After training, the weight of each patient was distributed almost evenly between the each patient's legs and this distribution was not significantly different from that of control subjects. Use of the force platform in this design required the training to be done in a laboratory setting. While some of the systems used to operate and analyze force platform data could be simplified, there remains a degree of complexity in running the training sessions and interpreting the results. Accordingly, the use of force platforms is primarily limited to the laboratory.

In another approach, patients with bilateral vestibular loss were trained using a mechanically non-supportive surface. The patients placed their index finger on the surface while attempting to maintain a quiet stance with their eyes closed. This method was effective in improving balance by reducing sway. This method is simple, can be used in nearly any situation and doesn't require any specific equipment. However, this method does not provide any feedback or indication of conditions which may be indicative of an impending fall and, accordingly, is not well suited for use as an analytical tool for determining the presence of pre-fall conditions.

In another approach, a group of patients were trained using a virtual reality head mounted display. The patients walked on a treadmill while the display showed virtual obstacles of various sizes. The patients were provided with vibro-tactile feedback when the patient's foot made virtual contact with the virtual obstacle. The training group showed an increased ability to negotiate a real obstacle course after three virtual reality training sessions.

Based on the foregoing, it appears that the key to improving balance in those individuals with deficiencies is dependent on the ability to accurately measure balance or the lack of balance. Over the years, measurements of postural sway using force platforms and optical tracking systems have been used to distinguish potential falters from non-fallers. Force platforms have been used to measure the center of pressure, which is the ground reaction force acting on the platform. While standing there are no other forces acting on the force platform. Therefore the ground reaction force is equivalent to the force applied to the platform due to the surface of the feet. This distributed force can be represented as a point force or pressure applied at the center of the force platform. Force platforms have been used to evaluate subjects standing quietly, standing on foam that is placed on the platform, while leaning anteriorly or posteriorly, standing with various stances, standing on platforms of changing length, standing on perturbed platforms, standing on rotating platforms, and portions of gait. Force platform testing has also been used to evaluate the effects of having eyes open and closed, rotating visual references, blurred vision, limited fields of view, medical conditions, and healthy elderly versus healthy young balance or posture characteristics. Optical tracking systems have been used to obtain measurements of the motion of the whole body center of mass as a balance indicator. Numerous studies of human movement using force platforms and optical tracking systems have been performed and have produced a wealth of information on various factors that affect balance.

From the results of these studies, it is clear that healthy elderly individuals sway more than healthy young persons, elderly with falling problems sway more than healthy elderly persons in both the sagittal and frontal planes, and even healthy subjects have more movement in the sagittal plane than the frontal plane. Also, as the width of stance increases, the sway changes very little in the sagittal plane, but decreases considerably in the frontal plane. Results also show that sway distance, velocity, and acceleration are greater in the sagittal plane than in the frontal plane, even when a wider, more stable stance is taken.

Force platforms and video-based motion tracking systems have been used in various forms since the 1950s. As such, force platforms and optical tracking systems have become the accepted standard for balance measurements. However, these methods can realistically only be used in a laboratory setting. Accordingly, systems and methods are needed which can be used to accurately measure sway distance, velocity, and acceleration in many settings, such as the settings encountered in every day life and not just the settings artificially constructed in the laboratory. This requires that the measurement system be low cost, easily moveable, small in size, adaptable to various body types and sizes, and compatible with activities of daily living.

### SUMMARY OF THE INVENTION

In one embodiment, a system for collecting data related to the angular orientation of a patient for use in determining the presence of pre-fall conditions includes a wireless sensor and a base unit. The wireless sensor may be attached to the patient and include a first microprocessor operatively coupled to at least one gyroscope and a transmitter. The base unit may include a second microprocessor operatively coupled to a receiver and a data storage unit. The gyroscope may output a signal related to the angular orientation of the patient to the first microprocessor. The first microprocessor processes the signal received from the gyroscope and transmits data related to the angular orientation of the patient via the transmitter. The receiver in the base unit receives the data related to the angular orientation of the patient from the wireless sensor and passes the data to the second microprocessor. The second microprocessor formats the data and stores the data in the data storage unit for analysis and a determination as to whether the data indicates the presence of pre-fall conditions.

### BRIEF DESCRIPTION OF THE DRAWINGS

The following detailed description of specific embodiments of the present invention can be understood when read in conjunction with the following drawings, where like structure is indicated with like reference numerals and in which:

FIG. 1 is a schematic diagram showing a system of detecting the angular orientation of a patient according to one embodiment shown and described herein;

FIG. 2 is a schematic diagram of a printed circuit board for attaching the gyroscope and pulse generator according to one embodiment of the wireless data acquisition system shown and described herein;

FIG. 3 is a photograph of the printed circuit board of FIG. 2;

FIG. 4 is a photograph of an RF transmitter for use in conjunction with the sensing unit of the wireless data acquisition (WDA) system shown and described herein;

FIG. 5 is a photograph of the assembled wireless sensor according to one embodiment of the WDA system shown and described herein;

FIG. 6 is a table showing computer code which may be used by the microprocessor of the wireless sensor to receive a signal from the gyroscope, process the signal, and transmit data derived from the signal according to one embodiment of the WDA system shown and described herein;

FIG. 7 is a table showing computer code which may be used by the microprocessor of the base unit to receive data from the wireless sensor, format the received data, and store the formatted data in a data storage unit;

FIG. 8 is a schematic diagram of the pulse generator, specifically a 556 integrated circuit timer, according to one embodiment of the WDA system shown and described herein;

FIG. 9 is a table showing the equations governing the operation of the 556 integrated circuit timer used as a pulse generator according to one embodiment of the WDA system shown and described herein;

FIG. 10. is a photograph of a base unit according to one embodiment of the WDA system shown and described herein;

FIG. 11 is a photograph showing a balance evaluation setup for determining pre-fall signatures using the WDA system according to one embodiment shown and described herein;

FIG. 12 is a photograph showing an experimental setup for determining pre-fall signatures using the WDA system according to one embodiment shown and described herein;

FIG. 13 is a chart showing the baseline or normal sway of a patient collected using the WDA system shown and described herein using the balance evaluation setup depicted in FIGS. 11 and 12;

FIG. 14 is a chart showing the baseline or normal sway of a human subject on the left and the sway of the human subject prior to a fall on the right collected using the WDA system shown and described herein. The pattern of sway prior to a fall may be used to define a pre-fall signature which may be subsequently used by the WDA system to provide the subject with a warning of the presence of pre-fall conditions.

### DETAILED DESCRIPTION OF THE ILLUSTRATIVE EMBODIMENTS

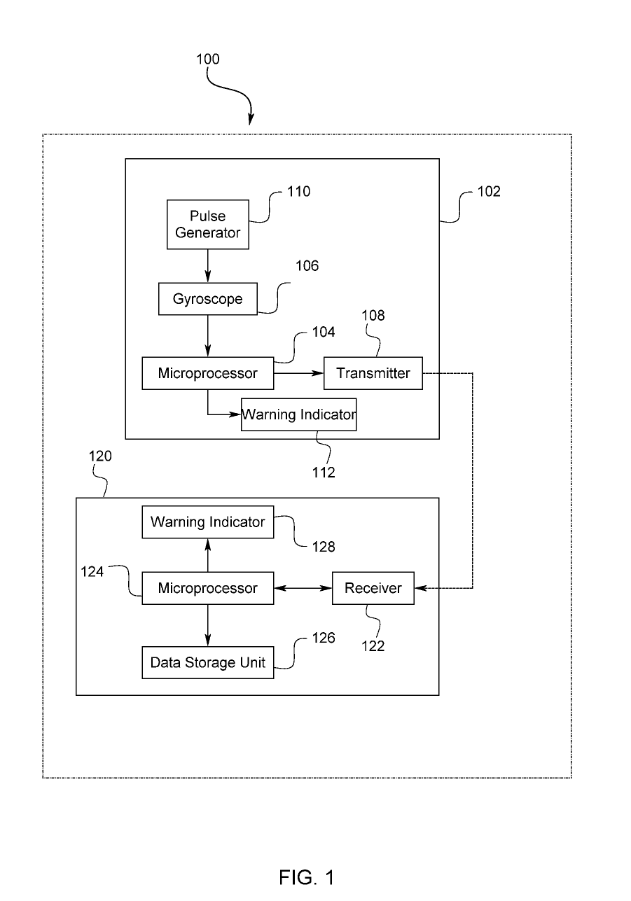

FIG. 1 depicts a schematic diagram of one embodiment of a wireless data acquisition system 100 for collecting data related to the angular orientation of a patient for use in determining if pre-fall conditions are present. The wireless data acquisition (WDA) system may generally comprise a wireless sensor and a base unit. The various elements that comprise the wireless sensor and the base unit of the WDA system, as well as methods for using both the WDA system and data collected from the WDA system, will be described in more detail herein.

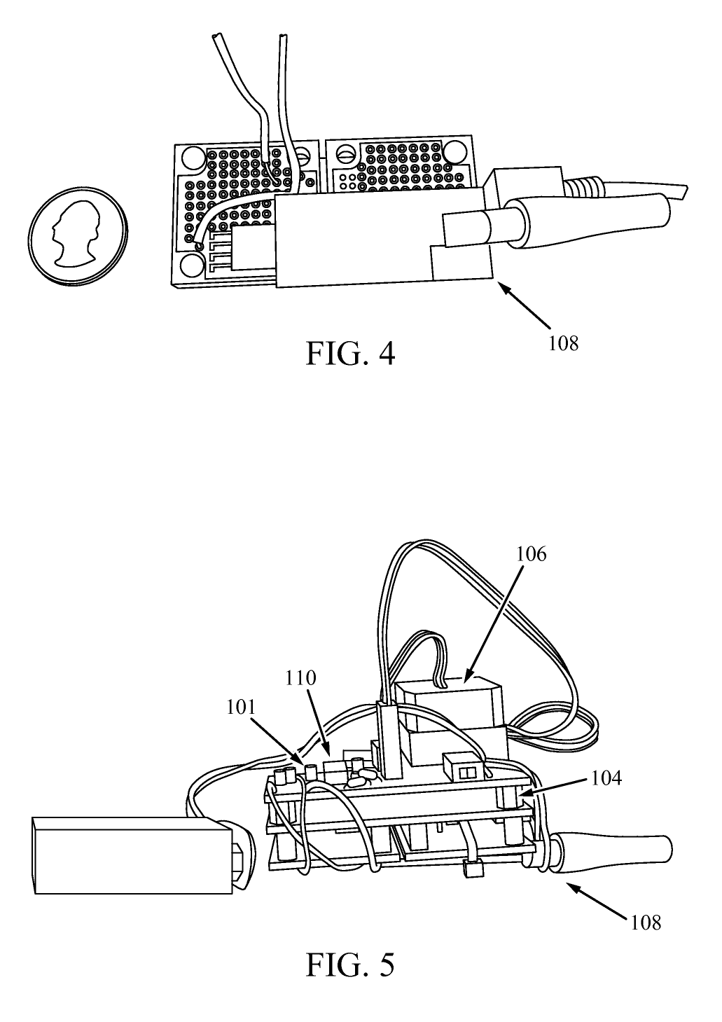

Referring now to FIGS. 1-5, the wireless sensor 102 of the WDA system is a small, wearable device capable of measuring and transmitting data related to the angular orientation of the body. The transmitted data may be used to determine postural sway during daily activities and identify conditions which may be indicative of or a signature of an imminent fall. The wireless sensor is a compact, portable, gyroscope-based system capable of real-time motion data acquisition and analysis which accurately and effectively measures postural sway.

The wireless sensor 102 may generally comprise a microprocessor 104, a gyroscope 106 and a transmitter 108. The microprocessor is operatively coupled to the gyroscope 106 and the transmitter 108 as shown in FIG. 1. The wireless sensor 102 may also comprise a pulse generator 110 operatively coupled to the gyroscope 106. In one embodiment, as shown in FIG. 1, the wireless sensor 102 may also comprise a warning indicator 112 for providing the user of the wireless sensor 102 with an audible, visual and/or a vibro-tactile indication of the presence of pre-fall conditions. Each of these components and their interconnectivity will be described in more detail below. Further, it should be understood that, while the arrows shown in FIG. 1 generally indicate the interconnectivity between various component parts of the WDA system, it should also be understood that these arrows are also indicative of electrical signals passed between the various component parts of the WDA system 100.

The microprocessor 104 is programmed to receive signals from the gyroscope 106 relating to the orientation of the gyroscope, process the signals, and pass data derived from the gyroscope signals to the transmitter 108. In one embodiment, the microprocessor 104 may also be programmed to filter the data derived from the gyroscope signals and thereby minimize and/or optimize the amount of data that is transmitted by the transmitter 108. For example, in one embodiment, the microprocessor may be programmed to filter data contained within a pre-determined exclusion band while data outside of the exclusion band is passed to the transmitter for transmission. The exclusion band may contain data corresponding to baseline or normal gyroscope orientations while the transmitted data corresponds to gyroscope orientations which depart from the baseline orientations and, therefore, may be indicative of a potential fall on pre-fall conditions. In another embodiment, the microprocessor may be programmed to process data received from the gyroscope 106 relating to the orientation of the gyroscope and, based on the processed data, determine the presence of conditions indicative of an impending fall. When such pre-fall conditions are present, the microprocessor 104 may also be programmed to activate the warning indicator 112 thereby providing the user with an audible, visual, or vibro-tactile indicator of the presence of such pre-fall conditions.

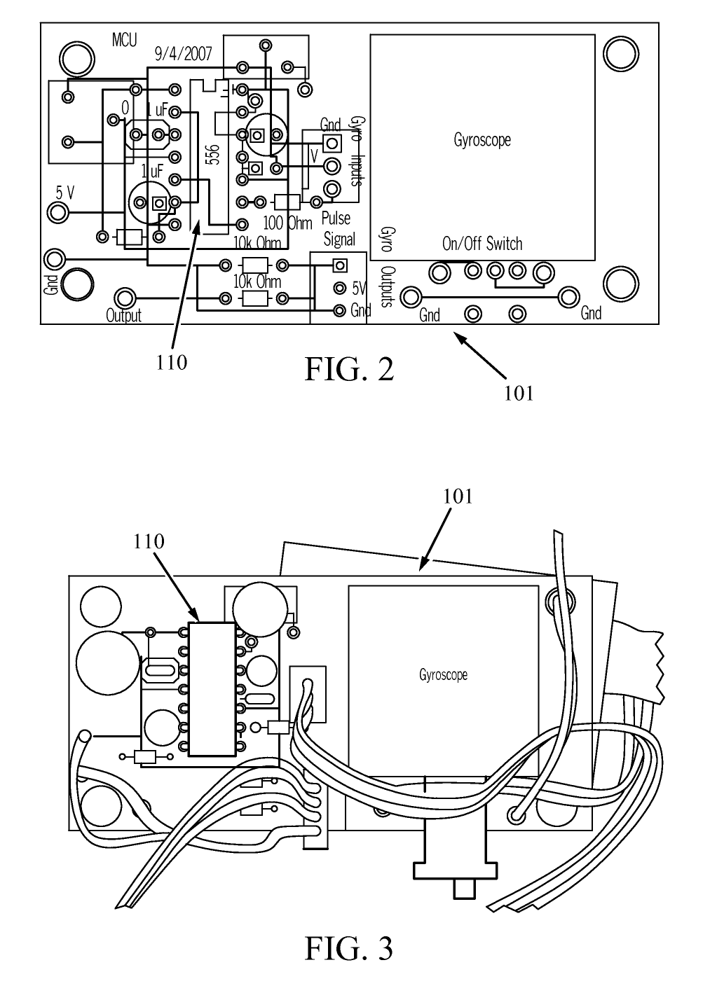

In one embodiment the microprocessor 104 of the wireless sensor 102 may comprise a BS2pe BASIC Stamp SX microcontroller mounted on a BASIC Stamp 2pe motherboard. Both the microprocessor and motherboard are manufactured by Parallax, Inc. The BASIC Stamp 2pe motherboard includes a USB port for interfacing with a computer and thereby facilitate programming of the microprocessor. Additional circuitry may be attached to the microprocessor using prototyping daughterboards such as the BASIC Stamp 2pe Proto-DB prototyping daughterboard manufactured by Parallax, Inc. The prototyping daughterboards may be attached to the motherboard via the daughterboard sockets. For example, in the embodiment shown in FIGS. 2 and 3, the gyroscope 106 and the transmitter 108 may be attached to the motherboard and microprocessor using the BASIC Stamp 2pe Proto-DB prototyping daughterboards. More specifically, the daughterboard B slot may operatively couple the gyroscope to the microprocessor by connecting pin seven of the motherboard to daughterboard pin C, which is, in turn, connected to the gyroscope output. The transmitter may be similarly connected to the motherboard using pin five of the motherboard to connect with daughterboard pin D, which, in turn, is connected to the input pin of the transmitter. A portable power supply, such as a battery, may be used in conjunction with the power supply connection of the motherboard to power the motherboard, which, in turn, provides a regulated output of five volts DC to the daughterboard thereby powering the various components attached to the daughterboard. While specific reference has been made herein to the microprocessor 104 being a Parallax BS2pe BASIC Stamp SX microcontroller, it should be understood that the microprocessor 104 may be any microprocessor suitable for receiving a signal from a gyroscope, processing the signal and passing data derived from the signal to a transmitter for transmission as will be apparent to one skilled in the art.

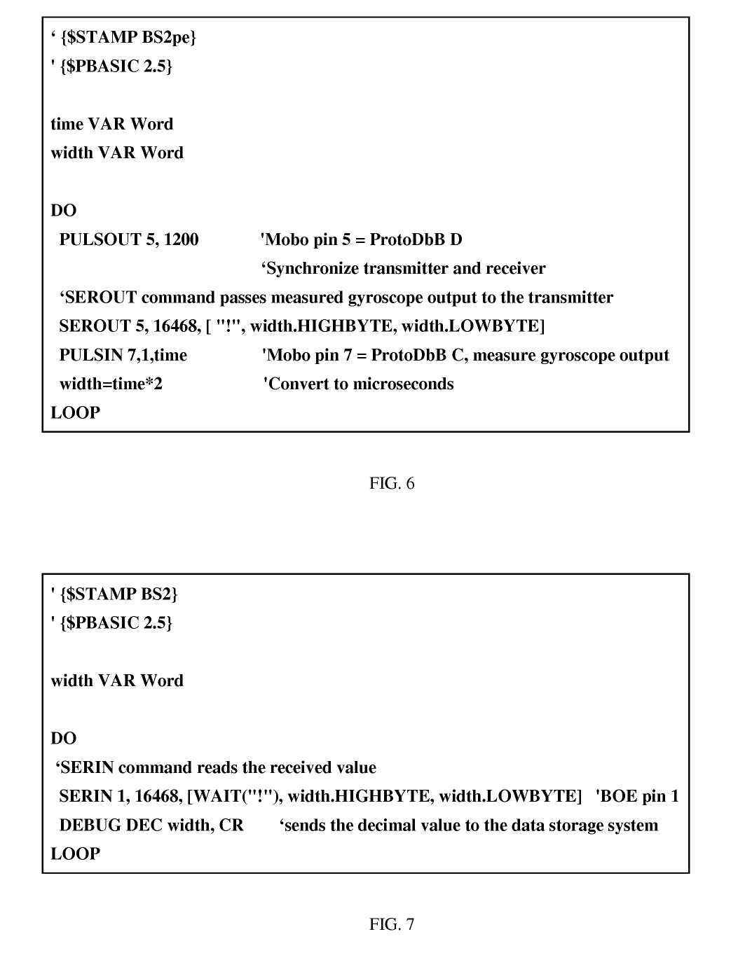

As discussed hereinabove, the microprocessor 104 may be programmed to receive a signal from the gyroscope and measure the pulse width of the signal. The pulse width of the gyroscope output signal is indicative of the angular orientation of the gyroscope 106. When the microprocessor 104 is a BS2pe BASIC Stamp SX microcontroller mounted on a BASIC Stamp 2pe motherboard, the microprocessor 104 may be programmed with the PBasic programming language using the Basic Stamp editor from Parallax, Inc. The program may utilize the PULSIN, PULSOUT, and SEROUT functions of the PBasic language to receive the signal from the gyroscope 106, process the signal, and pass data to the transmitter 108. Specifically, the PULSIN function may be used to measure the pulse width of the signal received from the gyroscope 106 and store the width as a variable. The PULSOUT function may be used to synchronize the transmitter 108 with a receiver before data is sent. The SEROUT function may be used to send data related to the pulse width of the signal received from the gyroscope 106 to the transmitter 108. Microprocessor code which may facilitate the receipt, processing and transmission of data is shown at FIG. 6. While the code depicted in FIG. 6 represents one embodiment of code which may be used to facilitate the receipt, processing and transmission of data by the microprocessor 104, it should be understood that the receipt, processing, and transmission of data by the microprocessor 104 of the sensor unit 102 may be performed using a different code or logic structure as will be apparent to one skilled in the art.

The gyroscope 106 used in the wireless sensor 102 may comprise an electronic gyroscope such as a micro-electromechanical-system (MEMS)-based, heading hold gyroscope. The heading hold feature of the gyroscope eliminates drift in the output signal of the gyroscope when the gyroscope is maintained in a non-zero angular orientation for an extended period of time. Non-heading hold gyroscopes require the use of complex software filters and/or additional hardware to compensate for this signal drift. Accordingly, the use of a heading hold gyroscope presents a significant advantage over non-heading hold gyroscopes as the heading hold feature eliminates signal drift in the output signal of the gyroscope and, therefore, eliminates the need for complicated hardware and/or software filters for conditioning and correcting drift in the output signal of the gyroscope 106. Further, the use of a MEMS-based gyroscope reduces or mitigates temperature effects which adversely impact other types of gyroscope devices such as piezoelectric gyroscopes and mechanical gyroscopes.

In one embodiment, the gyroscope 106 is a Futaba GY240 gyroscope. The GY240 utilizes a MEMS sensor to detect the angular orientation of the gyroscope and includes an angular vector control system (AVCS) to facilitate heading hold functionality. The AVCS of the gyroscope may be switched on or off via a mechanical switch and the sensitivity of the AVCS may be adjusted using a potentiometer. When used in the wireless sensor 102, the AVCS of the gyroscope is switched on and the sensitivity is set to approximately 75% of the maximum sensitivity. The output signal from the GY240 is indicative of the angular orientation of the gyroscope 106 and therefore the angular orientation of the wireless sensor 102. While specific reference is made herein to the use of a MEMS-based heading hold gyroscope such as the Futaba GY240, it should be understood that any electronic gyroscope having heading hold functionality may be used as will be apparent to one skilled in the art.

The electronic gyroscopes 106 used in the wireless sensor 102 are generally suitable for measuring the angular orientation of the wireless sensor 102 about one rotational axis. As discussed further herein, the gyroscope 106 may be removably attached to the printed circuit board 101 with hook and loop closures to facilitate repositioning the gyroscope 106 on the printed circuit board 101. When the gyroscope 106 is repositioned on the printed circuit board 101, the angular orientation of the gyroscope 106 with respect to the printed circuit board 101 may be adjusted such that the re-positioned gyroscope 106 is oriented to measure the angular orientation of the wireless sensor 102 about a different rotational axis. Accordingly, the ability to reposition the gyroscope 106 on the printed circuit board 101 facilitates measuring the angular orientation of the wireless sensor 102 about different rotational axes.

In another embodiment, the wireless sensor 102 may comprise two or more electronic gyroscopes for measuring the rotational orientation of the wireless sensor 102 about multiple rotational axes. In one embodiment, the gyroscopes may be oriented along mutually perpendicular axes to facilitate measuring the angular orientation of the wireless sensor on each of the axes.

The gyroscope 106 requires an input pulse to trigger a measurement of the angular orientation of the gyroscope 106 which, in turn, produces an output signal from the gyroscope 106 corresponding to the measured angular orientation of the gyroscope 106. Accordingly, to facilitate the measurement of the angular orientation of the gyroscope 106 at regular intervals, a pulse generator 110 may be connected to the input of the gyroscope 106. The pulse generator 110 may be configured to input pulses into the gyroscope 106 at regular intervals such that the angular orientation of the gyroscope 106 is determined and passed to the output of the gyroscope 106 at regular intervals.

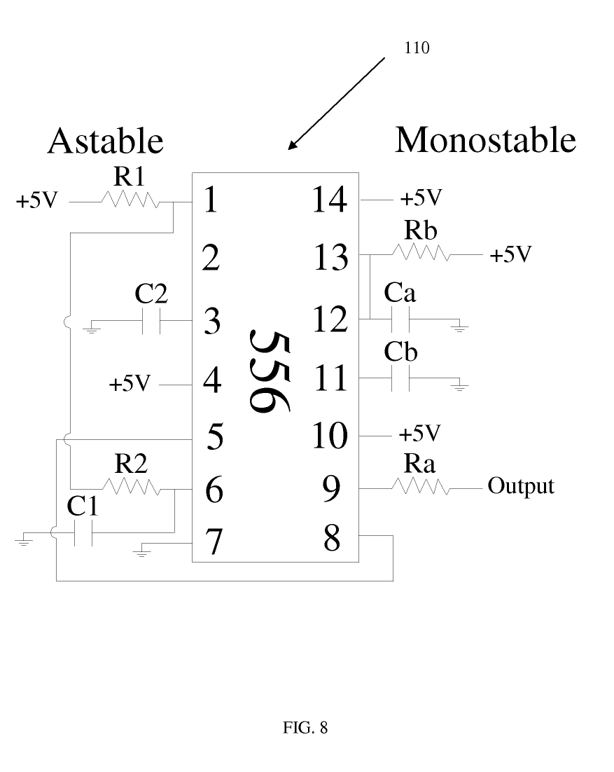

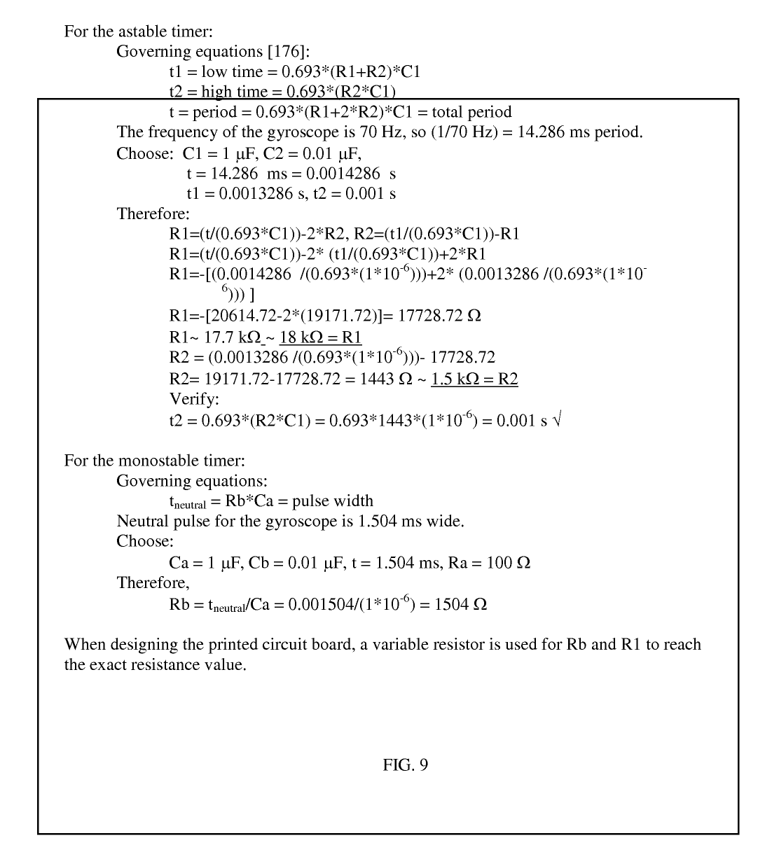

In one embodiment, the pulse generator 110 is an integrated circuit timer such as a general purpose 556 timer circuit. The 556 timer is connected to the input of the gyroscope 106 and configured to provide the gyroscope 106 with an input pulse to trigger the angular orientation measurement at regular intervals. Where the gyroscope is a GY240 gyroscope, the 556 timer may be configured to provide a 1.5 ms-wide output pulse to the input of the GY240 gyroscope at a frequency of 70 Hz. The frequency and width of the output pulse are dictated by the input requirements of the GY240 gyroscope. A wiring diagram for the 556 pulse generator is shown in FIG. 8. The equations governing the output of the pulse generator are shown in FIG. 9.

As shown in FIGS. 2 and 3, a custom printed circuit board 101 was manufactured to facilitate the connection of the 556 timer to the GY240 gyroscope. The custom printed circuit board 101 provides the various connections and holds the various electrical components which facilitate operation of the gyroscope 106 in the desired manner. The pulse generator 110, in this case the 556 timer, and associated electrical components are fixedly attached to the custom printed circuit board 101 while the GY240 gyroscope is removably attached to the custom printed circuit board with hook and loop fasteners to facilitate repositioning of the gyroscope 106 as needed. As discussed hereinabove, the custom printed circuit board 101 containing the gyroscope 106 and the pulse generator 110 is connected to the microprocessor 104 via prototyping daughterboards.

As discussed herein, the microprocessor 104 may be operatively coupled to a transmitter 108. In one embodiment, the transmitter 108 may comprise a transmitter/receiver or transceiver such that the wireless sensor 102 is enabled to both send and receive data. In another embodiment, the transmitter is an RF transmitter such as the 433 MHz RF Transmitter manufactured by Parallax, Inc. The transmitter 108 is operatively coupled to the microprocessor 104 via a prototyping daughter board as discussed hereinabove. The transmitter 108 may be capable of data transfer rates in the range from about 12,000-19.2K baud depending on the programming of the attached microprocessor. The transmitter may have an effective range of up to about 500 feet depending on the environmental conditions. While specific reference has been made herein to an RF transmitter, it should be understood that any transmitter suitable for sending a data signal may be used as will be apparent to one skilled in the art.

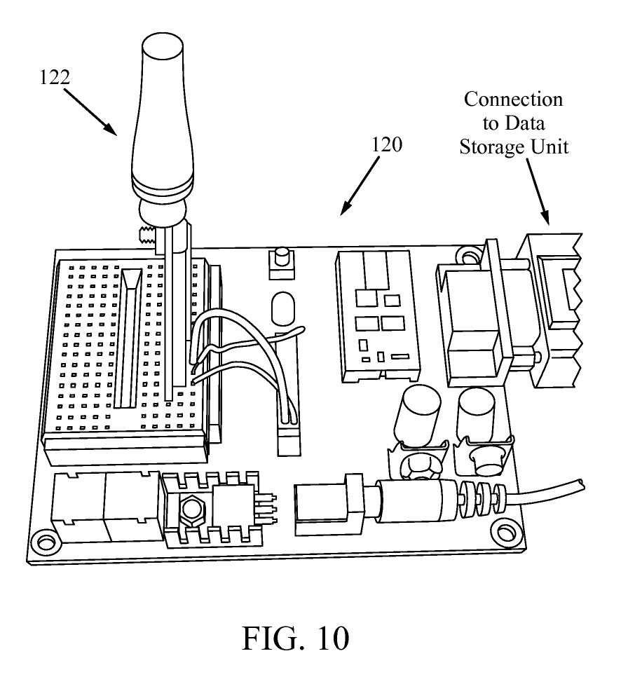

In addition to the wireless sensor 102, the WDA system 100 may also comprise a base unit 120 for receiving, formatting, and storing data transmitted from the wireless sensor 102. Referring now to FIGS. 1 and 10, the base unit 120 may generally comprise a receiver 122, a microprocessor 124, and a data storage unit 126 as shown in FIG. 1. The microprocessor 124 is operatively coupled to the receiver 122 and the data storage unit 126 as indicated in FIG. 1. The base unit 120 may also comprise a warning indicator 128, such as a visual or audible warning indicator, operatively coupled to the microprocessor 124 as shown in FIG. 1.

The receiver 122 receives a data signal transmitted from the wireless sensor 102 as indicated by the dashed arrow shown in FIG. 1. In one embodiment, the receiver 122 may comprise a transmitter/receiver or a transceiver such that the base unit 120 is enabled to both send and receive data to and from the wireless sensor 102. In another embodiment, the receiver 122 may comprise a 433 MHz RF receiver manufactured by Parallax, Inc. having a data transfer rate of about 12,000- to about 19.2K dependent on the programming of the associated microprocessor 124. The receiver may be operatively coupled to the microprocessor 124 as will be discussed further herein. While specific reference is made herein to the receiver being an RF receiver, any wireless receiver capable of receiving a transmitted data signal may be used as will be apparent to one skilled in the art.

The microprocessor 124 of the base unit 120 may be programmed to receive data from the receiver, format the data, and pass the data to a data storage unit. In one embodiment, the microprocessor 124 may be a Basic Stamp 2 microcontroller mounted on a Board of Education printed circuit board, both of which are manufactured by Parallax, Inc. The microprocessor 124 may be programmed to receive data from the receiver 122, format the data, and pass the data on to a data storage system. The receiver may be connected to the prototyping breadboard of the printed circuit board which is, in turn, connected to the input/output sockets of the Basic Stamp 2 microcontroller. While specific reference has been made herein to the microprocessor 124 being a Basic Stamp 2 microcontroller, it should be understood that the microprocessor 124 may be any microprocessor suitable for receiving, formatting and transmitting data to a data storage device.

In one embodiment, where the microprocessor 124 comprises the Basic Stamp 2 microcontroller, the microprocessor may be programmed to receive, process and transmit data with the PBASIC programming language using the Basic Stamp editor. More specifically, the microprocessor may be programmed with the SERIN and DEBUG functions of the PBASIC programming language. The SERIN function reads the data passed from the receiver. The DEBUG formats the data to a decimal value and sends the data to the data storage system. PBASIC code which may facilitate the receipt, processing and transmission of data is shown at FIG. 7. However, it should be understood that other codes may be used to achieve the same functionality as will be apparent to one skilled in the art.

After processing, the data received by the base unit 120 is stored in a data storage unit 126. The data storage unit 126 may comprise a hard drive, flash memory, a computer, a server or a similar data storage device as will be apparent to one skilled in the art. In one embodiment, the data storage unit 126 may be integral with the base unit 120 as shown in FIG. 1. In another embodiment, the data storage unit 126 may comprise an external data storage unit such as a computer operatively coupled to the base unit 120. For example, the microprocessor 124 of the base unit 120 may be linked to a personal computer using an RS-232 cable as shown in FIG. 10. Software loaded on the computer, such as Windows HyperTerminal, may be used to enable data transfer between the microprocessor and the computer. The data received by the computer may be stored as a text file.

Referring to FIG. 1, the WDA system may generally operate in the following manner: the pulse generator 110 sends a 1.5 ms pulse to the gyroscope 106 at a frequency of 70 Hz. Each pulse received by the gyroscope 106 triggers the gyroscope to determine the angular orientation of the gyroscope and produce an output signal corresponding to the angular orientation. Specifically, the width of pulses contained in the output signal correspond to the angular orientation of the MEMS sensor of the gyroscope 106. The output signal of the gyroscope 106 is received by the microprocessor 104 where the signal is processed and the width of each pulse in the signal is determined. The microprocessor 104 passes data corresponding to the width of each output pulse in the gyroscope 106 output signal to the transmitter 108 which transmits the data to the receiver 122 of the base unit 120. The receiver 122 of the base unit 120 receives the transmitted data and passes the data to the microprocessor 124 of the base unit. The microprocessor 124 converts the data into decimal format and sends the data to the data storage unit 126 from where the data may be retrieved, analyzed, displayed, etc.

While reference has been made herein to the use of specific components in constructing the sensor unit and the base unit of the WDA system, it should be understood that various other commercially available components may be substituted for the specifically identified components while still preserving the functionality of the sensor unit, the base unit, and the overall WDA system, as will be apparent to one skilled in the art.

The WDA system 100 has particular applicability in determining and collecting data relating to the angular orientation of a patient. This data may then be used to identify postural perturbations or angular orientations of the patient that are indicative of or a signature of an impending fall. Thereafter, the WDA system 100 may be programmed to analyze the data related to the angular orientation of the patient in real time and, upon identifying the presence of pre-fall conditions, activate a warning indicator thereby providing the user with an indication of a pending fall.

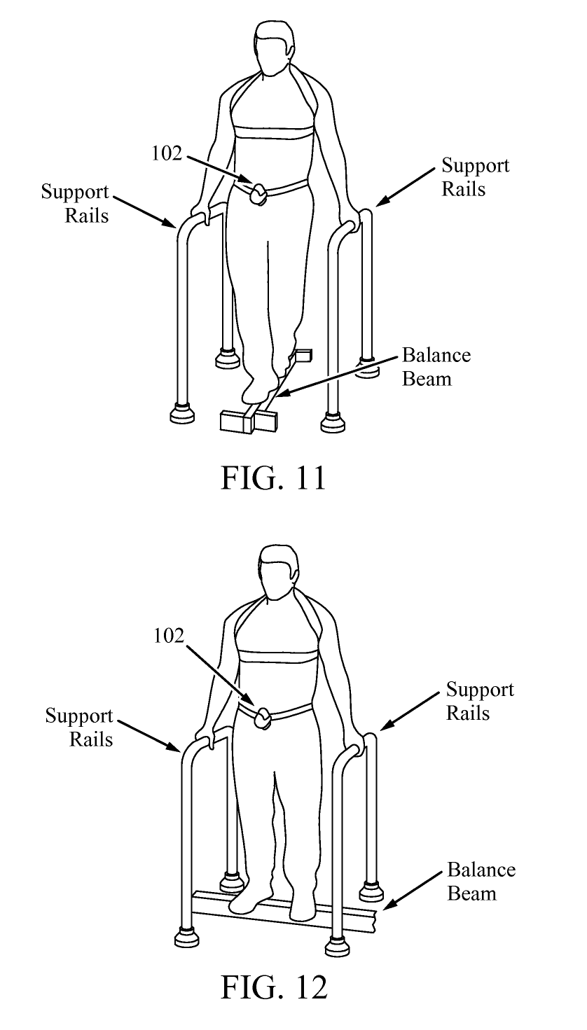

In one embodiment, the WDA system is first used to identify a pattern of data or an electronic signature produced by the WDA system indicative of pre-fall conditions for a particular patient. To accomplish this, the wireless sensor of the WDA system is attached to the patient and positioned such that the wireless sensor measures the sway of the patient relative to a particular plane through the patient's body. The wireless sensor 102 of the WDA system may be attached to the patient using hook and loop fasteners and straps attached to the wireless sensor. In order to measure the sway of the patient, the wireless sensor 102 may be attached to the torso at hip level on the anterior side of the subject, as shown in FIGS. 11 and 12. Alternatively, the wireless sensor 102 may be attached around the upper torso just under the arms with the wireless sensor 102 resting on the sternum, on the subject's dominant side shoulder, or on top of the head.

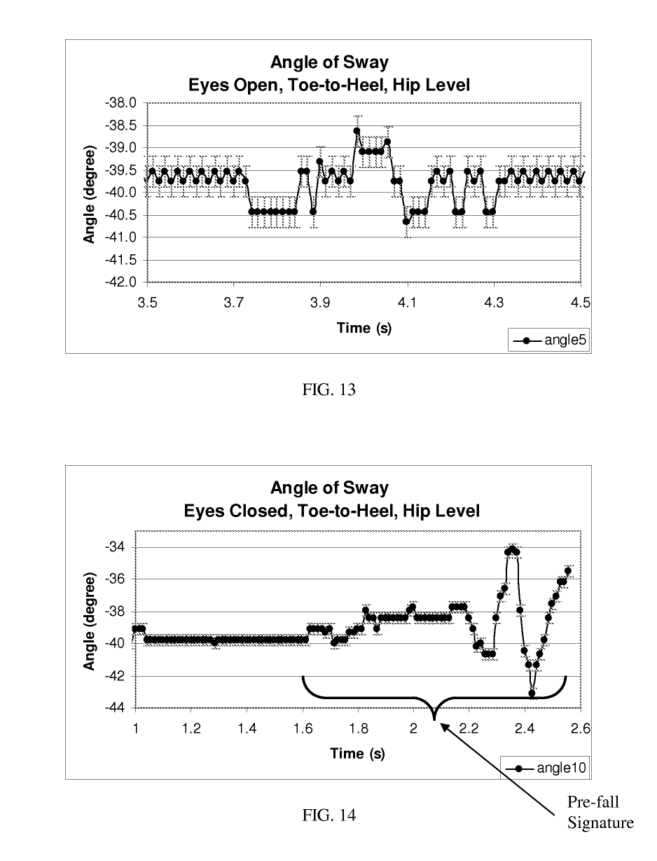

With the wireless sensor firmly attached to the patient any motion of the patient in the plane parallel to the rotational axis of a gyroscope is measured by the wireless sensor and a signal corresponding to the angular orientation of the gyroscope, and therefore the angular orientation of the patient, is outputted to the base unit where the signal is converted to a decimal format and stored in the data storage unit as a function of time. FIG. 13 shows an exemplary output signal captured by the WDA system described herein. The signal is indicative of normal or baseline sway of a patient wearing the wireless sensor.

With the wireless sensor in place, the patient may perform a variety of balance related tasks or tests under controlled, laboratory conditions. For example, the patient's balance may be evaluated using methods such as those disclosed by Bara et al. in the publication entitled “Increasing cognitive load with increasing balance challenge: recipe for catastrophe,” Exp. Brain Res., 174, 734-745 (2006), which is incorporated herein by reference. Using this method, falls may be induced under controlled conditions by placing the patient on a balance beam located on the floor and having the patient perform various mental tasks. A “fall” is defined as any time the patient touches support rails positioned on either side of the balance beam to keep from stepping off the balance beam. The test may be performed with the patient's eyes open and/or closed and with the subject's feet positioned parallel to the length of the balance beam in a heel to toe manner or with the subjects feet positioned perpendicular to the length of the balance beam at a shoulder width apart, as shown in FIGS. 11 and 12. Data is collected and stored by the WDA system throughout the balance evaluation.

It should be understood that the balance evaluation method used, specifically the method described in the publication by Bara et al., is one specific embodiment of a method for testing and/or evaluating balance by inducing falls. One skilled in the art will recognize that other methods of inducing falls may be used in conjunction with the WDA system shown and described herein to assess the balance of a patient and establish signatures indicative of pre-fall conditions for the patient.

In one embodiment, the data collected during the balance evaluation may be collected and stored in the data storage unit for later analysis and a determination or identification of pre-fall signatures. In another embodiment, the data collected from the WDA system during the balance evaluation is analyzed and displayed in real time using a computer and/or software operatively linked to the base unit. The computer may be an integral part of the base unit such as when the data storage unit is a computer. Alternatively, the computer may be operatively linked to the base unit via a wired or wireless connection such that data stored in the data storage unit may be uploaded to the computer for analysis. The computer may visually display the data in a chart or graph as shown in FIGS. 13 and 14 where the angle of sway (e.g., the angle of orientation of the patient) of the patient is displayed as a function of time.

Referring specifically to FIGS. 13 and 14, data related to the angular disorientation of the patient is shown as a function of time. This data may be used to determine a signature indicative of the presence of pre-fall conditions by correlating the data with the falls induced during the balance evaluation. For example, the collected data displayed in FIG. 13 is representative of the baseline or normal sway of the patient without an induced fall. However, the collected data displayed in FIG. 14, specifically the data displayed on the right side of the chart, shows the sway of the patient due to an induced fall. Accordingly, this data is representative of the sway (e.g., the angular orientation of the patient) just prior to a fall and may be used determine one or more pre-fall signatures indicative of the angular orientation of the patient just prior to a fall. This pre-fall signature may then be used in conjunction with the WDA system to determine the presence of pre-fall conditions based on the angular orientation of the patient.

For example, in one embodiment, after the pre-fall signature is determined, the microprocessor in the wireless sensor may be programmed to compare data derived from the output signal of the gyroscope to the pre-fall signature. If the data matches the pre-fall signature, the wireless sensor may be programmed to activate the warning indicator thereby providing the patient with a visual, audible and/or vibro-tactile warning of the presence of pre-fall conditions. Alternatively or additionally, the wireless sensor unit may also be programmed to send a signal to the base unit indicative of the presence of the pre-fall conditions. Upon receipt of the signal, the microprocessor in the base unit may be programmed to activate a warning indicator operatively associated with the base unit and/or issue a request for assistance via an attached computer having an internet, network, phone connections, or the like.

In another embodiment, after the pre-fall signature is determined, the microprocessor in the base unit may be programmed to compare data derived from the output signal of the gyroscope to the pre-fall signature. If the data matches the pre-fall signature the base unit may be programmed to activate a warning indicator operatively attached to the base unit. The warning indicator may be visual, audible and/or a combination thereof. In another embodiment, when the transmitter of the wireless sensor and the receiver of the base unit are transceivers, the base unit may send a signal to the wireless sensor indicating the presence of pre-fall conditions. Upon receipt of the signal from the base unit indicating the presence of pre-fall conditions, the wireless sensor may be programmed to actuate a warning indicator thereby providing the patient with a visual, audible and/or vibro-tactile response indicating the presence of pre-fall conditions.

Accordingly, the WDA system shown herein may be used to perform several different functions. For instance, the WDA system may be used as an analytical tool for determining the relationship between the angular orientation of a patient and balance. In this role, the WDA system facilitates the collection and real time analysis of data related to the angular orientation of the patient as the patient performs routine tasks or is subjected to a balance evaluation. The collected data may be used as feedback for training the patient to improve balance and mitigate potential injuries caused by the lack of balance. Moreover, because the WDA system is compact and portable, and because the wireless sensor is wearable, the WDA system may be used to monitor patients and collect data outside of the artificial environment of the laboratory. For example, the WDA system may be easily installed in a patient's home and used to monitor the patient's balance during the routine activities of daily life. The collected data may be used to identify potentially dangerous behaviors and conditions which could cause injury due to the lack or loss of balance.

The WDA system may also be used as a diagnostic tool to identify and/or diagnose various medical conditions which result in a loss of balance. For example the WDA system may be used to diagnose or identify balance disorders due to localized or generalized muscle weakness, medical or neuropsychiatric conditions, the side effects of medication, and the like.

Further, the WDA system may also be used as a safety device to prevent or reduce the occurrence of falls. As discussed herein, the WDA system may be used to study the balance of a patient and identify pre-fall signatures in the angular orientation of the patient which are indicative of a pending fall. This information can then be used in conjunction with the WDA system to identify the presence of these pre-fall signatures and provide the patient with a warning or other indication of the presence of such pre-fall conditions. The warning permits the patient to modify his or her behavior and/or posture and thereby mitigate the risk of falling.

It will be apparent to those skilled in the art that various modifications and variations may be made to the present invention without departing from the spirit and scope of the invention. Thus it is intended that the present invention cover the modifications and variations of this invention provided they come within the scope of the appended claims and their equivalents.

What is claimed is:

1. A system for collecting data related to the angular orientation of a patient for use in determining if pre-fall conditions are present, the system comprising:
a wireless sensor for attachment to the subject comprising a first microprocessor operatively coupled to at least one gyroscope and a transmitter, wherein the gyroscope outputs a signal relating to the angular orientation of the patient to the first microprocessor and wherein the first microprocessor processes the signal received from the gyroscope and transmits data related to the angular orientation of the patient via the transmitter; and
a base unit comprising a second microprocessor operatively coupled to a receiver and a data storage unit, wherein the receiver receives data related to the angular orientation of the patient from the wireless sensor and passes the data to the second microprocessor which formats the data and stores the data in the data storage unit for analysis and a determination as to whether the data indicates the presence of pre-fall conditions.
